(19) United States
(12) Patent Application Publication
(10) Pub. No.: US 2024/0122869 A1
(43) Pub. Date: Apr. 18, 2024

(54) WOUND HEALING MEANS, METHOD OF MANUFACTURE THEREOF AND USE THEREOF

(71) Applicant: CONTIPRO A.S., Dolni Dobrouc (CZ)

(72) Inventors: Kristyna SKUHROVCOVA, Praha 9 (CZ); Adela KOTZIANOVA, Opava, Katerinky (CZ); Katerina KNOTKOVA, Chocen (CZ); Vladimir VELEBNY, Zamberk (CZ)

(73) Assignee: CONTIPRO A.S., Dolni Dobrouc (CZ)

(21) Appl. No.: 18/263,037

(22) PCT Filed: Jan. 25, 2022
(86) PCT No.: PCT/CZ2022/050006

§ 371 Date: Jul. 26, 2023

(30) Foreign Application Priority Data
Jan. 26, 2021 (CZ) PV2021-32

(51) Int. Cl. A61K 9/70; A61K 47/36; A61L 15/28; A61L 15/44

(52) U.S. Cl. CPC A61K 9/70; A61K 47/36; A61L 15/28; A61L 15/44; A61L 2400/12

(57) ABSTRACT

The present invention relates to wound healing means comprising nanofibrous carrier based on two types of hyaluronic acid derivatives, the photocurable derivative of HA and the hydrophobized derivative of HA or the pharmaceutically acceptable salts thereof and which combine to form a mechanically resistant nanofibrous structure that is stable in aqueous solutions. The invention further relates to a method of manufacture of such means and use thereof.

### FIELD OF INVENTION

The present invention relates to a wound healing means comprising a nanofibrous material based on two types of hyaluronic acid derivatives, i.e. a photocurable hyaluronic acid derivative and a hydrophobized hyaluronic acid derivative or pharmaceutically acceptable salts thereof, a combination of which forming a mechanically resistant nanofibrous structure that is stable in aqueous solutions. The invention further relates to a process of the manufacture of such means and use thereof.

### BACKGROUND OF THE INVENTION

Synthetic and natural polymers have been used as basic materials for the preparation of nanofibrous materials. Nanofibers, usually in the form of thin layers, can be prepared by electrospinning from a variety of synthetic and natural polymers. This method, i.e. spinning of polymer solutions, was described previously in patent documents, for example U.S. Pat. Nos. 4,043,331 and 5,522,879. Nowadays, these materials are widely used in biomedicine and their fields of application include, for example, tissue engineering (U.S. Pat. No. 10,653,635), drug distribution (ES 2 690 483) and wound healing (WO 2 016 059 611).

The use of nanofibrous materials is particularly advantageous in topical applications, i.e. in the treatment of skin and soft tissue damage—the structure of the nanofibrous materials resembles a fibrous structure formed by naturally occurring collagen commonly present in the extracellular matrix. The materials available for these topical applications, i.e. wound dressings, come in various forms (e.g. gauze, films, foams), but must always meet certain criteria. The ideal cover should keep the wound clean and sufficiently moist while draining and absorbing the excess exudate produced by the wound. The cover should prevent the penetration of microorganisms and unwanted particles. The cover must be permeable at the same time to allow gas exchange. Last but not least, its application must be simple and painless, where the cover should keep its shape and not disturb the wound, especially during removal (for example, it must not stick to the wound). The publication [1] compared the morphological and physical properties of various types of absorbent wound dressings available today (hydrocolloids, alginates and foams). The pore size in these materials is in the order of hundreds of μm. Only the alginate cover had a fibrous structure. This type of cover degrades after a few hours, the fibrous structure disappears and a compact gel forms. It has been shown that the absorption capacity depends on the number of pores, when the highly porous fibrous alginate cover showed the highest absorption capacity (swelling 2000%/12 hours), however, it is limited by the degradation time.

Hydrocolloid covers showed low absorption capacity (swelling ≤400%/12 hours) and slow absorption of exudate. In order to prevent tissue maceration, an appropriate ratio between the absorption capacity and the degree of dehydration has to be achieved, which is aided by the slower absorption of exudate and the sufficient penetration of water vapor through the cover, which prevents the accumulation of exudate. Hydrocolloid covers showed insufficient permeability for water vapor, the best results were achieved with a fibrous alginate cover. Numerous studies have shown that nanofibrous materials are suitable for use as wound dressings [2, 3, 4]. One of their main advantages is that the structure of the nanofiber layer facilitates cell proliferation and re-epithelialization of tissues and improves non-specific protein adhesion, which is the first step in activating the immune response cascade and initiating the healing process. The use of nanofiber wound dressings therefore prevents undesired prolongation of the healing time, which is essential in the treatment of chronic wounds in particular [5]. In addition, the pores between the individual fibers are small enough to prevent infiltration of microorganisms into the wound and cause infection, but large enough to make the material permeable [6].

In these applications, the basic—primarily hydrophobic—synthetic polymer performs a rather mechanical function (PV 2014-674), while the added natural polymer shows biological activity. An example is the Czech patent application PV 2018-537 concerning the preparation of a preparation for healing skin defects, where the preparation consists of polyesters and their copolymers (according to examples polylactic acid, polyhydroxybutyrate or polycaprolactone) and biologically active components (here platelets) are incorporated in the next step. The disadvantage of the preparation thus prepared is the need to use toxic solvents for the preparation of spinning solutions and preparation in a two-step process. In addition, the polymers used are highly hydrophobic and may not result in sufficient exudate removal. Another example may be utility model 31723, the technical solution of which relates to the cover of an acute or chronic wound. Here, the combination of polycaprolactone and polylactic acid is used to form a nano- and microfibrous wound dressing with the advantage that the resulting dressing will not need to be removed from the wound due to its degradation. The porous structure should ensure sufficient gas exchange, removal of metabolites from the wound and maintain a suitable climate at the wound (UM does not include data to confirm this). The disadvantage of this solution is again mainly the non-wettability of both polymers, when sufficient exudate removal will not be achieved and a sufficiently humid environment for healing will not be created. Similar results were obtained when measuring the contact angle of nanofibrous materials from the above-mentioned polylactic acid, polycaprolactone and polycaprolactone composite, where the materials were evaluated as highly hydrophobic and less wettable, but the addition of hydrophilic, natural gelatin achieved higher layer absorbency [7]. Another disadvantage will be the degradability over a period of many weeks, which is not necessary especially in the case of acute wounds, which may affect the release of any incorporated active substances and they may be released too late.

One of the natural polymers with significant biological activity is hyaluronic acid (HA or hyaluronan). It is a linear glycosaminoglycan comprising regularly alternating units of D-glucuronic acid and N-acetyl-D-glucosamine. HA is a natural component of tissues and plays an important role in processes such as hydration or healing. Due to its biocompatibility, biodegradability and non-toxicity, it is used in many not only medical applications. HA is used for the preparation of nanofibrous materials. It is added either as a gel-forming additive component (see CZ patent 308285), or it is possible to prepare nanofibers directly from it or its modified derivatives. Nanofibers consisting of pure native HA are prepared using mainly organic solvents or acids, for example, CN patent document 101775704 or publications [15], [16] and [17]. Within CN 101775704, nanofibers of HA (Mw 400 to 2,000,000 g/mol) were prepared by electrostatic spinning from a solvent system of formic acid and dimethylformamide, i.e. from highly toxic solvents. Ideally, the electrospinning process ensures complete evaporation of the solvents, however, instabilities of the process can cause insufficient evaporation and consequently the presence of solvents in the prepared material. The use of less toxic solvents is thus an advantage in medical applications. The HA nanofibers thus prepared are immediately soluble in aqueous solutions. Another example may be CZ patent 308492 relating to a cosmetic composition based on hyaluronic acid nanofibers. In this case, HA is spun from water together with a synthetic hydrophilic polymer, which is referred to as a carrier polymer (polyethylene oxide or polyvinyl alcohol), the content of this carrier polymer being from 15 to 99 wt. %. The nanofibers are prepared here from an aqueous solution and without the carrier polymer the spinning process would not be feasible, and the higher the proportion of synthetic polymer, the higher the yield of the whole process. The cosmetic preparation further contains active substances, the HA content in the dry matter is thus between 2 and 90 wt. %. The nanofiber cosmetic preparation thus prepared is also highly hydrophilic and thus immediately soluble in aqueous solutions, which is desirable for said cosmetic application. Similarly, nanofibers of HA and synthetic hydrophilic polymer have been prepared, for example, in [8], [9], [10] or [11]. Due to its high hydrophilicity, native HA and the nanofibers prepared from it are not suitable in applications where a longer lasting effect is required, such as wound dressing, nevertheless, it is the strong hydrophilic nature of native hyaluronic acid and its ability to bind water in its structure, that makes it a very promising material for so called moist wound healing. In addition, native HA, although a component of connective tissues that are naturally stressed during movement, does not exhibit the high mechanical strength required for this type of application in nanofiber form. Therefore, synthetic hydrophobic polymers that are not completely soluble in water are also used to prepare nanofibrous materials containing native HA. In these cases, HA is usually in a minor amount and after contact with the aqueous solution it is washed out, the resulting nanofibrous material has after washing HA properties defined by the selected synthetic polymer (e.g. [12], [13], [14], [25], [26]). However, the most predominantly hydrophobic synthetic polymers have a long degradation time and to complete dissolution require the presence of organic solvents that are toxic and can trigger strictly undesirable depolymerizations when preparing the hyaluronan spinning solution [27]. It is therefore advantageous to maintain the composition of the nanofiber layer primarily on the basis of a modified natural polymer, which is in a relative majority (at least 95 wt. %) compared to the synthetic polymer. This can be achieved by covalent crosslinking of HA, which, however, is often accompanied by the presence of toxic crosslinking agents such as divinyl sulfone, glutaraldehyde or butane-1,4-diol diglycidyl ether (e.g. [18], [19]) or by the formation of HA derivatives. Whereas the type of derivative defines the final properties of the materials prepared from it.

The preparation of nanofibrous materials from HA derivatives is very unique. An example is publication [20], where thiolated HA (T-HA, Mw HA 1,500,000 g/mol) was used. The T-HA was then spinning together with polyethylene oxide (PEO, Mw 900,000 g/mol, Dulbecco's Modified Eagle's Medium solvent, T-HA/PEO ratio 4:1 and 1:1) and a crosslinking agent. The PEO was subsequently washed out with water after crosslinking the layer. Another example is the publication [21], in which the authors focused on the use of lightcurable methacrylated HA (M-HA) with a conjugated RGD peptide. The fiber mixture consisted of synthesized M-HA, PEO (Mw 900,000 g/mol) and the photoinitiator Irgacure 2959 all dissolved in water. A stable fibrous structure was achieved in aqueous solutions. In the publication [22], the authors focus only marginally on the preparation of nanofibrous materials from furyl acryloyl HA (F-HA), in combination with hydrophilic PEO (80 wt. % F-HA, 20 wt. % PEO). The prepared F-HA/PEO nanofiber materials were crosslinked by UV irradiation for 5, 10 or 30 minutes. The publication shows the retention of the porous structure of the material after immersion in water, but does not state the time for which the material was soaked. The long-term stability in aqueous solutions of the prepared material is thus not known, nor are known its mechanical properties. Nanofibrous materials prepared from various photo-curable HA derivatives are also addressed in CZ patent 304 977. HA derivative is here spun together with carrier polymer (polyvinyl alcohol, polyacrylic acid, PEO or polyvinyl pyrrolidon) whose ratio in the final structure makes 50-99 wt. %, preferably 80 wt. %, the HA derivative is thus in preferable embodiment represented by only 20 wt. %. Stability is also achieved thanks to other biocompatible synthetic hydrophobic polymers and their copolymers (carboxymethyl cellulose, gelatin, chitosan, polycaprolactone, polylactic acid, polyamide, polyurethane, poly-(lactide-co-glycolic) acid). The mechanical robustness, which was not substantiated by data in this patent, is also attributed to the low absorbency of the prepared fibers (only about 20%). The preservation of the nanofiber structure after wetting has not been discussed here, only the SEM image after wetting is given, when the fiber structure is considerably degraded. The use of HA derivatives for the preparation of nanofibrous materials is also addressed by CZ patent 307 158, which again mentions HA, F-HA and HA derivatives containing a saturated or unsaturated C₃-C₂₁ chain, which do not require subsequent crosslinking. However, the subject-matter of this patent was to create a water-soluble nanofibrous material (drug carrier, solubility from 50 to 100% in 0.05 to 10 s), stability in an aqueous environment, mechanical properties and preservation of the nanofibrous structure were not subject-matter of this solution. Even in this case, it was spun in a mixture with PEO or polyvinyl alcohol. The content of the HA derivative in the nanofibrous material is in the range from 5 to 90 wt. %.

Water-stable nanofibrous materials can be achieved by using other natural polymers, for example, the publication presented highly hydrophobic nanofibrous materials consisting of a mixture of ethyl cellulose and zein, i.e. polymers that do not show essential biological activity. The material was developed as a carrier for active substances. The disadvantage of the use of synthetic polymers is also a considerable environmental burden, the use of natural polymers can prepare a water-stable fully degradable material. An example is the publication [24], in which nanofibrous materials were prepared from a mixture of polyvinyl alcohol, gluten and soy flour, the resulting material was crosslinked with non-toxic crosslinkers to increase hydrophobicity and resistance. In this case, the nanofiber structure was preserved, but after 1 day in the water, the nanofiber structure merged and the porosity was lost. Other examples are these documents combining natural polymers with synthetic ones [28, 29, 30]. In general, when mixed with a synthetic polymer, natural polymers contribute to higher wettability, and thus higher absorbent capacity, which is an important property of wound dressings.

As mentioned above, there are a number of criteria that a material suitable as a wound healing cover must meet. Available solutions include either compositions prepared mainly from synthetic polymers with insufficient absorption capacity and low permeability but meeting the requirements of mechanical stability or compositions prepared from natural polymers or combinations of natural and synthetic polymers providing sufficient absorption capacity and permeability, but with unsatisfactory mechanical parameters and too fast degradation. The need to use toxic solvents in the preparation and the often complex production of a given composition involving a multi-step process also appear to be problematic.

### SUMMARY OF THE INVENTION

The above-mentioned drawbacks are overcome by a wound healing means based on hyaluronic acid derivatives, the essence of which is to include nanofibers containing
a crosslinked photocurable ester of hyaluronic acid derivative or a pharmaceutically acceptable salt thereof, wherein at least two ester groups of a photocurable ester derivative of hyaluronic acid or a pharmaceutically acceptable salt thereof of general formula I

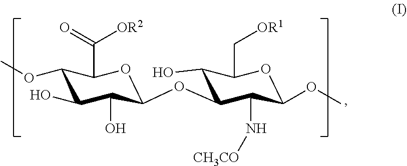

where
R¹ is independently H or COCHCH furyl,
R² is H⁺ or pharmaceutically acceptable salt thereof, and weight average molecular thereof weight is in the range 82 000 g/mol to 110 000 g/mol and degree of substitution thereof is in the range from 4 to 20%,
forms cyclobutane circle of general formula II,

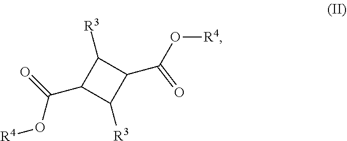

where R³ is furyl and
R⁴ is the main chain of hyaluronic acid or a pharmaceutically acceptable salt thereof,
hydrophobized hyaluronic acid derivative or a pharmaceutically acceptable salt thereof of general formula III,

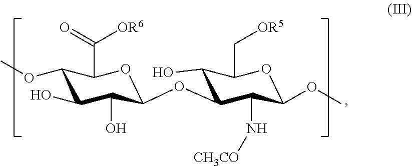

where
R⁵ is H or —C(═)C₁₂H₂₃,
R⁶ is H⁺ or pharmaceutically acceptable salt and weight average molecular weight thereof is in the range 300,000 g/mol to 350,000 g/mol and degree of substitution thereof is in the range from 65% to 95%,
and polyethylene oxide of weight average molecular weight in the range from 300,000 g/mol to 900,000 g/mol.

According to one embodiment of the means of the invention, the degree of substitution of the photocurable ester derivative of hyaluronic acid or a pharmaceutically acceptable salt thereof is preferably in the range of 5 to 10%, more preferably 5%. According to another embodiment of the means of the invention, the degree of substitution of the hydrophobized hyaluronic acid derivative or the pharmaceutically acceptable salt thereof ranges preferably from 65% to 80%, more preferably 73%. The weight average molecular weight of the polyethylene oxide is preferably from 400,000 g/mol to 600,000 g/mol, more preferably 600,000 g/mol.

According to another preferred embodiment of the means according to the invention, the nanofibers further comprise at least one active agent, which is either a biologically active agent and/or at least one diagnostic agent. The biologically active agent is selected from the group containing antibiotics, antiallergics, antifungals, antineoplastics, antiphlogistics, antivirals, antioxidants, or antiseptics or native hyaluronic acid or a pharmaceutically acceptable salt thereof, preferably the biologically active agent is selected from the group containing diclofenac, triclosan, octenidine, latanoprost, salicylic acid, gallic acid, ferulic acid, Ibuprofen, Naproxen, Cetirizine, quercetin, epicatechin, chrysin, luteolin, curcumin, ciprofloxacin. The diagnostic agent is preferably selected from the group containing Brilliant Green, Fluorescein Isocyanate, Curcumin or Methylene Blue.

According to another preferred embodiment of the means according to the invention, the content of the crosslinked photocured ester derivative of hyaluronic acid or a pharmaceutically acceptable salt thereof is from 15 wt. % to 75 wt. %, more preferably from 45 wt. % to 75 wt. %, the most preferably 48 wt. % relative to the total weight of nanofibers. It is a cross-linked ester of 3-(2-furyl)acrylic acid and hyaluronic acid or the pharmaceutically acceptable salt thereof (F-HA).

According to another preferred embodiment of the means according to the invention, the content of the hydrophobized hyaluronic acid derivative or the pharmaceutically acceptable salt thereof is from 15 wt. % to 75 wt. %, more preferably from 45 wt. % to 75 wt. %, the most preferably 48 wt. %, relative to the total weight of the nanofibers. It is an ester of lauric acid and hyaluronic acid or the pharmaceutically acceptable salt thereof (L-HA).

According to another preferred embodiment of the means according to the invention, the content of polyethylene oxide is in the range from 3.5 wt. % to 10 wt. %, more preferably from 4 wt. % to 5 wt. %, the most preferably 4 wt. %, relative to the total weight of the nanofibers.

According to another preferred embodiment of the means according to the invention, the active agent content is in the range from 0.01 to 10 wt. %, preferably from 0.1 to 5 wt. % relative to the total weight of nanofibers.

According to another preferred embodiment of the means according to the invention, the nanofibers have a diameter in the range from 100 nm to 1000 nm, preferably from 250 nm to 500 nm.

According to another preferred embodiment of the means according to the invention, it is in a form of a dry layer having an areal weight in the range from 1 to 100 g/m², preferably in the range from 1 to 20 g/m², more preferably in the range from 10 to 15 g/m².

According to another preferred embodiment of the means according to the invention, absorption capacity thereof is in the range of 1000 to 3500%, more preferably 1500 to 2500%, at least 1 hour after wetting in aqueous solution.

According to another preferred embodiment of the means according to the invention, porosity thereof is maintained for 72 hours after wetting in aqueous solution.

In another aspect, means of the invention is prepared by first electrospinning a spinning solution comprising a mixture of water and a water-miscible polar solvent, a photocurable hyaluronic acid ester derivative or a pharmaceutically acceptable salt thereof of formula I, a hydrophobized hyaluronic acid derivative or a pharmaceutically acceptable salt thereof of formula III and polyethylene oxide, to form nanofibers, after which the resulting nanofibers are photocured by crosslinking a photocurable hyaluronic acid ester derivative or a pharmaceutically acceptable salt thereof of general formula I by irradiation in the UV wavelength range. The water-miscible polar solvent is preferably isopropanol.

According to another preferred embodiment of the method of manufacture of the means according to the invention, the water content in the spinning solution is in the range of 30 to 50 vol. %, more preferably 50 vol. % and the water-miscible polar solvent is in the range of 50 to 70 vol. %, more preferably 50 vol. % to the total volume of the spinning solution.

According to another preferred embodiment of the method of manufacture of the means according to the invention, the spinning solution preferably comprises distilled water and isopropyl alcohol.

According to another preferred embodiment of the method of manufacture of the means according to the invention, the spinning solution further comprises at least one active agent.

According to another preferred embodiment of the method of manufacture the means according to the invention, the spinning solution has a dry matter concentration of 2 to 5 wt. %, preferably 3 wt. %, where the proportion by weight in dry matter of
the photocurable ester derivative of hyaluronic acid or a pharmaceutically acceptable salt thereof according to the general formula I being from 15 wt. % to 75 wt. %, more preferably from 45 wt. % to 75 wt. %, the most preferably 48 wt. %,
the hydrophobized hyaluronic acid derivative or a pharmaceutically acceptable salt thereof according to the formula III being from 15 wt. % to 75 wt. %, more preferably from 45 wt. % to 75 wt. %, the most preferably 48 wt. %,
polyethylene oxide being in the range from 4 wt. % to 10 wt. %, more preferably from 4 wt. % to 5 wt. %, the most preferably 4 wt. %.

According to another preferred embodiment of the method for the preparation of the means according to the invention, the proportion by weight of active agent in the dry matter is in the range from 0.01 to 10 wt. %, preferably from 0.1 to 5 wt. %.

According to another preferred embodiment of the method of manufacture of the means according to the invention, the crosslinking by UV irradiation takes place for a period of 50 to 90 minutes, preferably 60 minutes.

According to another embodiment of the means according to the invention, it is intended for use in cosmetics, medicine or regenerative medicine, preferably in wound care, or on patches for external or internal use.

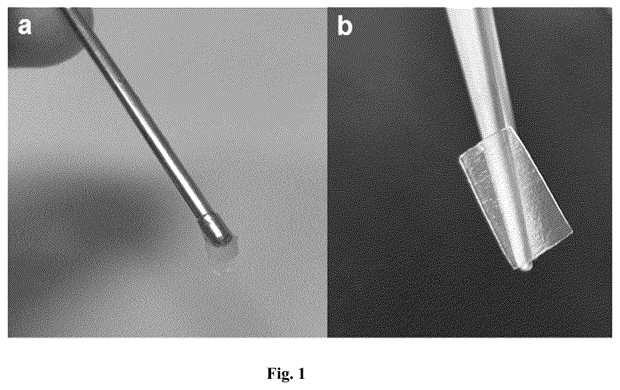

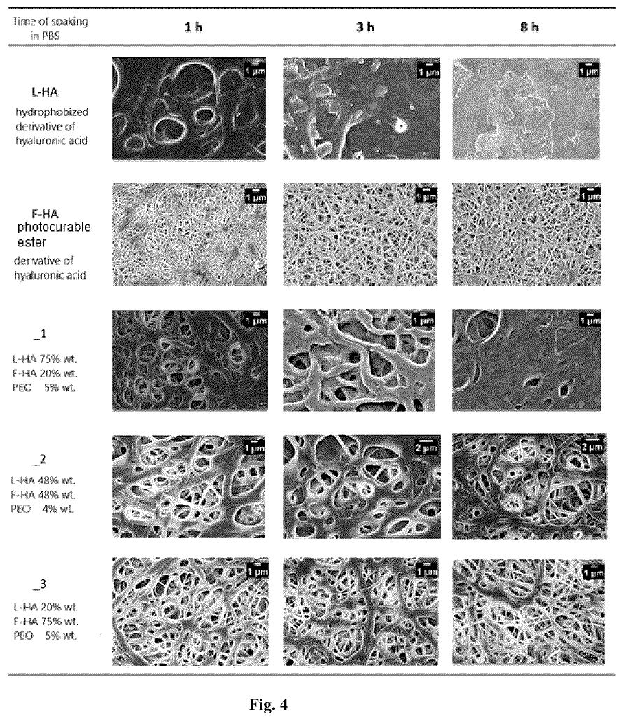

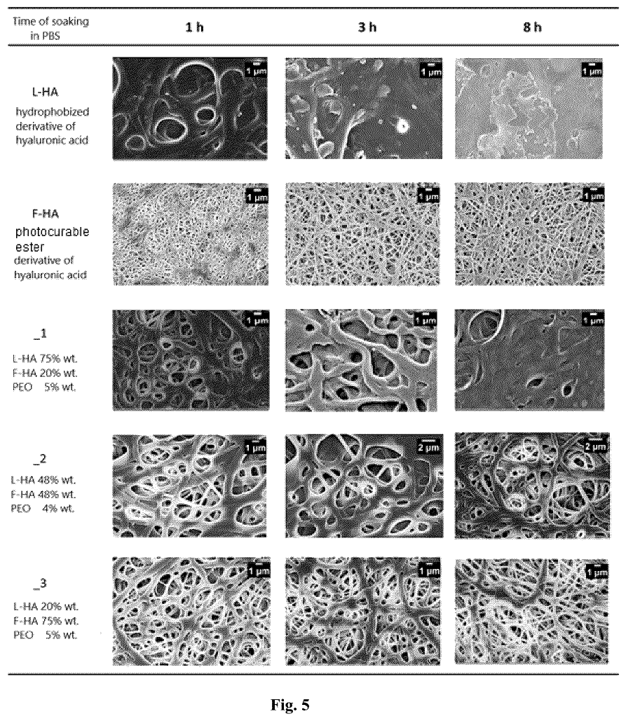

Nanofibrous materials prepared from the derivatives themselves do not show suitable properties for the given application—the HA derivative (F-HA) crosslinked by photo-hardening after wetting retains the nanofibrous structure but does not have suitable mechanical properties (see FIG. 1a, FIG. 4, FIG. 5), nanofibers only from the hydrophobized HA derivative fuse substantially immediately after wetting into a mechanically stable, compact film without pores (see FIG. 1b, FIG. 4, FIG. 5). The means according to the invention has been achieved just by combining these two hyaluronic acid derivatives. The means according to the invention excels in high stability in aqueous solutions, which can be advantageously used in the field of medical devices (e.g. dressing and wound healing). Stabilization of the means according to the invention does not require the presence of initiators or activators. The prepared material allows high absorption of aqueous solutions into structure thereof, with simultaneous structural, shape and mechanical stability, after absorption the nanofibrous material of gel-like structure is suitable for wet healing. The aqueous solution is preferably selected from the group containing saline, phosphate buffer (PBS) or TRIS buffer. The pH of the aqueous solution is typical of the wound's natural environment, which is usually a neutral to slightly basic pH in the range of 6 to 8.5. The means according to the invention is stable in this pH range.

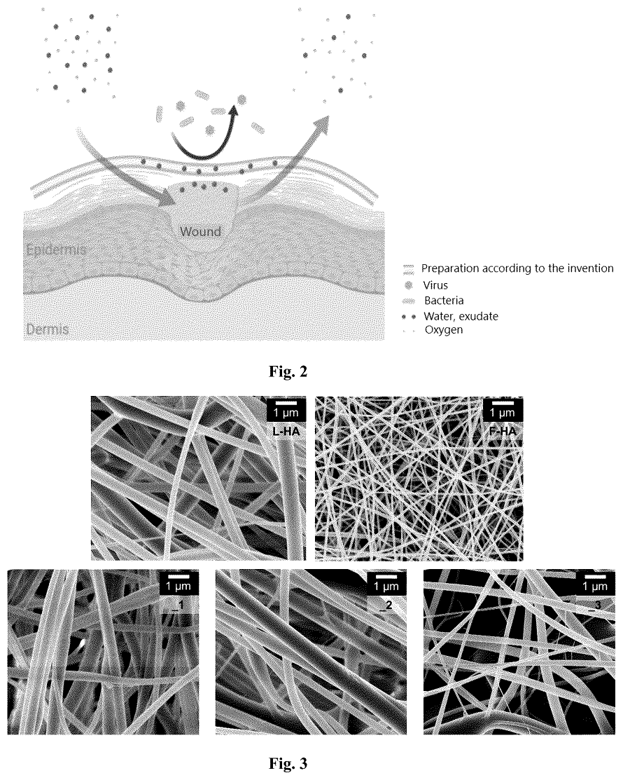

By structural stability is meant the preservation of the nanofibrous structure of the means according to the invention. This preservation of the nanofibrous structure even after wetting ensures sufficient porosity and thus permeability. At the same time, the size of the pores prevents the penetration of impurities, bacteria and viruses (FIG. 2). The weight ratio between used hyaluronic acid derivatives or pharmaceutically acceptable salts thereof defines the final properties of the prepared preparation, in particular the absorbency and permeability, which allows the preparation of the means for different types of wounds according to the amount of exudate. The means of the invention may advantageously contain one or more active agents that are released from the nanofibrous material upon absorption of the fluid. These substances can be preferably chosen from the group of hydrophilic and hydrophobic active agents, since the nanofibrous material is preferably prepared in a solvent mixture of distilled water and isopropyl alcohol, so that preparation under mild reaction conditions is also advantageous.

The above-described wound healing means according to the present invention is in the form of one or more nanofiber layers and exhibits advantageous properties over prior art means.
1) The means according to the present invention retains fibrous structure thereof for at least 1 hour after soaking in water or the aqueous solution.
2) The means according to the present invention retains a porous structure for at least 72 hours after soaking in water or the aqueous solution.
3) The means according to the present invention achieves an absorption capacity of at least 1000% after one hour of soaking in water or the aqueous solution,
4) The means according to the present invention maintains a stable shape for at least 72 hours after complete soaking in water or the aqueous solution.

The nanofiber means according to the invention is prepared by the electrospinning method, namely in a one-step process, the hyaluronic acid derivatives according to the invention, polyethylene oxide and optional active agents are dissolved in a single solvent system. The solvent system consists of distilled water in a content of 30 to 50 wt. %, more preferably 50 wt. %, and isopropyl alcohol in a content of 50 to 70 wt. %, more preferably 50 wt. %.

The concentration of all dry matter in the electrospinning solution is in the range of 2 to 5 wt. %, more preferably 3 wt. %.

The nanofiber means according to the invention comprises nanofibers having a diameter of 200 nm to 1000 nm, more preferably from 250 nm to 500 nm, in both the dry and wet states. In the wet state, the fibrous structure is maintained for 1 hour after wetting and even longer, depending on the relative weight proportion of the HA derivative (F-HA) crosslinked by photocuring to the total weight of the nanofibers in the means according to the invention.

The nanofiber structure is considered to be preserved and stable in the aqueous solution environment if the individual fibers can be clearly distinguished in the SEM image. These fibers can have a larger diameter than the dry fibers.

The porous structure is formed when the fibers are no longer distinguishable, and yet there are measurable pores in the SEM image. These pores are formed by the gradual swelling of individual fibers.

The nanofiber means according to the invention is suitable for use in cosmetics, medicine or regenerative medicine, preferably in wound care, or as part of a patch or wound dressing for external or internal use. The advantage of this product is also the dry form, which guarantees long-term stability of the product without the need for preservatives.

The nanofiber means according to the present invention, although the nanofiber layer is self-supporting, is not expected to be directly applied. The nanofiber means according to the invention is preferably spun on a carrier fabric or foil, with which it can be applied to the site of action in the case of a coating, with the possibility of adding an absorbent layer. The material of the carrier fabric, film or absorbent layer is selected from the group containing polyester, cellulose, polyurethane, polypropylene, polyethylene, viscose, polyamide, cotton or mixtures thereof. In the case of a patch, the carrier fabric or foil is preferably a base pad. It also has an absorption function at the same time.

Thus, a preferred embodiment of the invention is a wound healing cover comprising at least one carrier layer, which is provided with at least one nanofiber layer of the means according to the invention. The carrier layer is a fabric, foil or cushion. The carrier layer material is selected from the group containing polyester, cellulose, polyurethane, polypropylene, polyethylene, viscose, polyamide, cotton, or mixtures thereof. After application of such a cover according to the invention, the nanofiber layer according to the invention adheres to the wound.

In the case of both the patch and the wound cover, it is advantageous to anchor the nanofiber means according to the invention between a standard contact inert mesh of textile material and a pad preferably based on viscose or polypropylene foil, which helps to absorb and drain excess fluids from the wound. After applying the means to the wound, the contact inert mesh is completely at the bottom, i.e. in direct contact with the wound, protecting the nanofiber layer from mechanical damage, tearing after contact with moisture in the wound.

Thus, an even more preferred embodiment of the invention is a wound healing cover which further comprises a contact inert mesh based on polyester or polyester silk resting on the nanofiber layer according to the invention.

### DEFINITION OF TERMS

The term “aqueous solution” means water based solution with a pH in the range of 6 to 8.5, preferably in the range of 7 to 8.

The term “albumin salt solution” means an aqueous solution containing 5.84 g of sodium chloride, 3.36 g of sodium bicarbonate, 0.29 g of potassium chloride, 0.28 g of calcium chloride, 33.00 g of bovine albumin and 1000 ml demineralized water.

The term “nanofiber material”, “nanofiber layer”, means a continuous layer containing statistically intertwined (nano)fibers with a diameter not exceeding 1000 nm.

The term “dry nanofiber layer” means a self-supporting material consisting of statistically intertwined (nano)fibers with moisture residues corresponding to the relative humidity in the laboratory environment at a temperature of 23-24° C.

The term “stability in aqueous solution” means the shape and structural (fibrous) stability of the nanofiber layer after wetting thereof and remaining in the aqueous medium for a given period of time. At the same time, the material retains porous character thereof.

The term “permeability” means the bilateral permeation of gas molecules (oxygen, carbon dioxide) and water vapor, which occur naturally both at the site of injury and in the environment.

By “biologically active agent” is meant an active additive, or a mixture thereof, which produces a pharmacological effect at the site of action or directly affects the treatment/healing process.

The term “hyaluronic acid derivative” means a compound derived from the basic skeleton of hyaluronic acid, resulting from the substitution of the hydrogen atom of the hydroxyl group on the C6 carbon of the N-acetyl-D-glucosamine unit with another functional group.

By “pharmaceutically acceptable salt of hyaluronic acid” is meant a compound derived by derivation from the basic skeleton of high purity hyaluronic acid. The salt consists of hyaluronan anions and a specific cation selected from the group containing sodium, potassium, calcium.

The term “wound” means the loss or disruption of the skin cover due to physical, mechanical or thermal damage, or due to pathophysiological disorders or any damage to anatomical or physiological functions. Preferably, it is a chronic wound.

The term “degree of substitution” means the rate of substituents in % in the HA derivative (F-HA or L-HA) per 100 dimers of hyaluronic acid or a pharmaceutically acceptable salt thereof.

The term “absorption capacity” means a defined volume of aqueous solution that the material absorbs into structure thereof per unit of time. It is determined as the difference between the weight gain of the sample in the aqueous solution environment and the weight of the sample in the dry state.

By “porosity” is meant a property of a material that contains a number of bounded pores, the volume of which corresponds to the amount of void space in the total volume of the material.

By “molecular weight” is meant the weight average molecular weight (Mw), which was determined by ¹H NMR spectroscopy and confirmed by size exclusion chromatography (SEC/GPC).

By “wound healing cover” is meant an application form of the cover, such as a wound cover or a patch.

The total weight of the nanofibers corresponds to the weight of dry matter.

### DETAILED DESCRIPTION OF DRAWINGS

FIG. 1 a—Photograph of the nanofiber layer from the F-HA derivative crosslinked by photocuring after soaking in the aqueous solution, the sample does not achieve the required mechanical properties; b—photograph of the nanofiber layer of hydrophobized derivative HA (L-HA) after soaking in aqueous solution, the sample achieves the required mechanical properties, but is not permeable.

FIG. 2 Scheme showing the use of a nanofiber means according to the invention after application to a wound.

FIG. 3 SEM images showing the dry nanofibrous means of the invention after spinning from a spinning solution comprising the photocurable hyaluronic acid ester derivative or the pharmaceutically acceptable salt thereof of formula I (F-HA), the hydrophobized hyaluronic acid derivative or the pharmaceutically acceptable salt thereof of formula II (L-HA) and polyethylene oxide (PEO) in various proportions _1) prepared as in Example 1 below; _2) prepared as in Example 2 below; _3) prepared as in Example 3 below; together with SEM images of nanofibrous materials always prepared from only one of the derivatives in a mixture with PEO in a proportion of 90 wt. % each of HA derivative to 10 wt. % PEO.

FIG. 4 SEM images showing the morphology of nanofibrous means after spinning from a spinning solution containing the photocurable hyaluronic acid ester derivative or the pharmaceutically acceptable salt thereof of formula I (F-HA), the hydrophobized hyaluronic acid derivative or the pharmaceutically acceptable salt thereof of formula II (L-HA) and polyethylene oxide (PEO) in different proportions according to the invention after soaking in phosphate buffer at different time intervals—1, 3 and 8 hours; together with SEM images of soaked nanofibrous materials always prepared from only one of the derivatives in a mixture with PEO in a proportion of 90 wt. % each of derivative to 10 wt. % PEO.

FIG. 5 SEM images showing the morphology of nanofiber means after spinning from a spinning solution containing the photocurable ester derivative of hyaluronic acid or the pharmaceutically acceptable salt thereof of formula I (F-HA), the hydrophobized derivative of hyaluronic acid or the pharmaceutically acceptable salt thereof of formula II (L-HA) and polyethylene oxide (PEO) in different proportions according to the invention (as shown in the figure and prepared according to Examples 1 to 3 below) after soaking in phosphate buffer at different time intervals—24, 48 and 72 hours; together with SEM images of soaked nanofibrous materials always prepared from only one of the HA derivatives (F-HA or L-HA) in a mixture with PEO in a proportion of 90 wt. % each of derivative to 10 wt. % PEO.

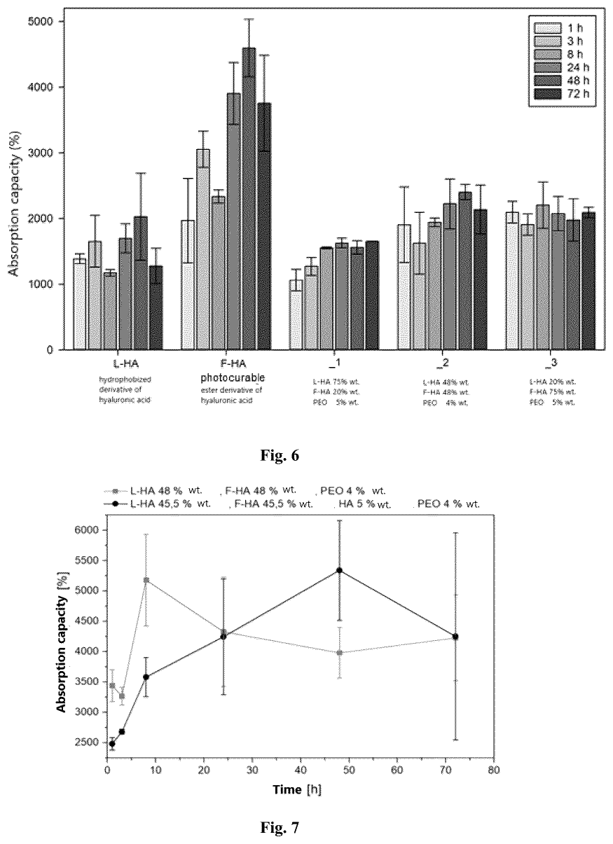

FIG. 6 Absorption capacity of prepared nanofibrous means from a spinning solution comprising the photocurable hyaluronic acid ester derivative or the pharmaceutically acceptable salt thereof of formula I (F-HA), the hydrophobized hyaluronic acid derivative or the pharmaceutically acceptable salt thereof of formula II (L-HA) and polyethylene oxide (PEO) in different proportions according to the invention (as shown in the figure and prepared according to Examples 1 to 3 below) after soaking in phosphate buffer at different time intervals—1, 3, 8, 24, 48 and 72 hours; together with the absorption capacity of nanofibrous materials prepared in each case from only one of the HA derivatives (F-HA or L-HA) in a mixture with PEO in a proportion of 90 wt. % each of derivative to 10 wt. % PEO soaked in PBS at the same time intervals.

FIG. 7 Absorption capacity of prepared nanofibrous means from a spinning solution containing the photocurable ester derivative of hyaluronic acid or the pharmaceutically acceptable salt thereof of formula I (F-HA, 48 wt. %), the hydrophobized derivative of hyaluronic acid or the pharmaceutically acceptable salt thereof of formula II (L-HA, 48 wt. %) and polyethylene oxide (PEO, 4 wt. %) according to the invention (as shown in the figure and prepared according to Example 2 below) and from a spinning solution containing the photocurable ester derivative of hyaluronic acid or the pharmaceutically acceptable salt of formula I (F-HA, 45.5 wt. %), the hydrophobized hyaluronic acid derivative or the pharmaceutically acceptable salt thereof of formula II (L-HA, 45.5 wt. %), native hyaluronic acid or the pharmaceutically acceptable salt thereof (HA, 5 wt. %) and polyethylene oxide (PEO, 4 wt. %) according to the invention after soaking in the albumin-containing salt solution at various time intervals—1, 3, 8, 24, 48 and 72 hours.

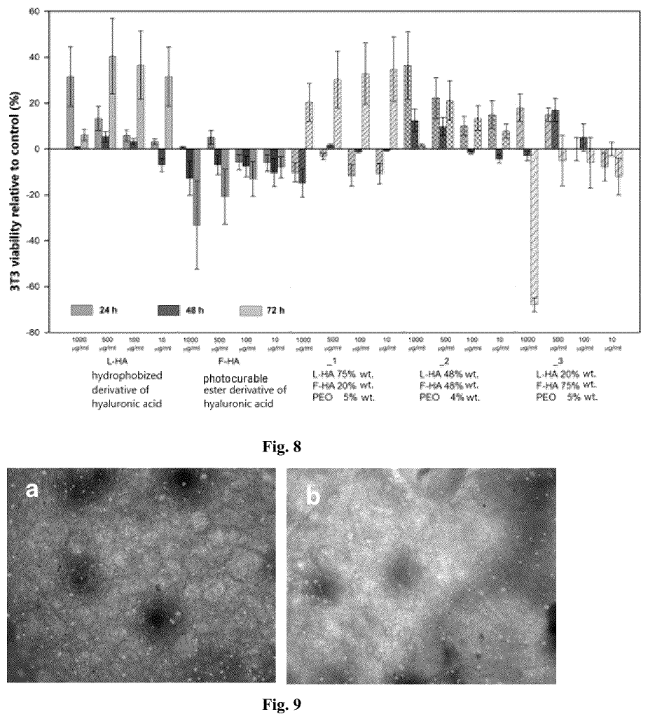

FIG. 8 Effect of different concentrations of prepared nanofibrous means from a spinning solution containing the photocurable ester derivative of hyaluronic acid or the pharmaceutically acceptable salt of formula I (F-HA), the hydrophobized derivative of hyaluronic acid or the pharmaceutically acceptable salt of formula II (L-HA) and polyethylene oxide (PEO) in various proportions according to the invention (as shown in the figure and prepared according to Examples 1 to 3 below) on the cell viability of 3T3 fibroblasts; together with the cell viability of nanofibrous materials prepared in each case from only one of the HA derivatives (F-HA or L-HA) in a mixture with PEO in a proportion of 90 wt. % each of derivative to 10 wt. % PEO.

FIG. 9 Fluorescence confocal microscopy images confirming the proadhesive ability of nanofibrous materials for cellular NHDF fibroblasts. Slide a shows a sample prepared according to Example 3 and slide b shows a sample prepared according to Example 2.

### EXAMPLES

For the preparation of the nanofiber layers listed below, hyaluronic acid derivatives prepared by Contipro a.s. using a 4SPIN LAB laboratory device (Contipro a.s.) were used.

### Example 1

An electrospinning solution was prepared using a 1:1 mixture of water and isopropyl alcohol as the solvent system. This solution further contained 75 wt. % of the hydrophobized hyaluronic acid derivative (L-HA, Mw 320,000 g/mol, DS 73%) or the pharmaceutically acceptable salt thereof, 20 wt. % of the photocurable ester derivative of hyaluronic acid (F-HA, Mw 98,000 g/mol, DS 5%) or the pharmaceutically acceptable salt thereof and 5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 55 kV, solution dosing 350 μL/min, electrode spacing 20 cm at a temperature of 20 to 25° C. and air humidity below 20% RH. In the method, a nanofiber layer with a weight of 11.11±1.29 g/m², a thickness of 15.77±2.46 μm and a fiber diameter of 304±106 nm was prepared. The prepared nanofiber layer is crosslinked for 60 minutes under UV irradiation at a wavelength of 302 nm. The nanofiber layer thus prepared has an absorption capacity of 1000%/1 hour, the maximum absorption capacity is reached when fully immersed in phosphate buffer (37° C.) in 8 hours and is 1500%. The nanofiber structure is maintained for 1 hour, then the fibers are swollen and fused, and after 72 hours a film with slightly preserved pores is formed. This type of material is especially suitable for less exuding wounds.

### Example 2

An electrospinning solution was prepared using a 1:1 mixture of water and isopropyl alcohol as the solvent system. This solution further contained 48 wt. % of the hydrophobized hyaluronic acid derivative (L-HA, Mw 320,000 g/mol, DS 73%) or the pharmaceutically acceptable salt thereof, 48 wt. % of a photocurable ester derivative of hyaluronic acid (F-HA, Mw 98,000 g/mol, DS 5%) or the pharmaceutically acceptable salt thereof and 4 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 55 kV, solution dosing 350 μL/min, electrode spacing 20 cm at a temperature of 20 to 25° C. and air humidity below 20% RH. In the process, a nanofiber layer with a weight of 7.29±0.43 g/m², a thickness of 11.75±0.89 μm and a fiber diameter of 479±230 nm was prepared. The prepared nanofiber layer is crosslinked for 60 minutes under UV irradiation at a wavelength of 302 nm. The nanofiber layer thus prepared has an absorption capacity of 1500%/1 hour, the maximum absorption capacity is reached when fully immersed in phosphate buffer (37° C.) in 8 hours and is 2000%. The nanofiber structure is maintained for 48 hours, followed by swelling and fusion of the fibers and enlarging of the pores. This type of material is especially suitable for more exuding wounds.

### Example 3

An electrospinning solution was prepared using a 1:1 mixture of water and isopropyl alcohol as the solvent system. This solution further contained 20 wt. % of the hydrophobized hyaluronic acid derivative (L-HA, Mw 320,000 g/mol, DS 73%) or the pharmaceutically acceptable salt thereof, 75 wt. % of a photocurable ester derivative of hyaluronic acid (F-HA, Mw 98,000 g/mol, DS 5%) or the pharmaceutically acceptable salt thereof and 5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 55 kV, solution dosing 350 μL/min, electrode spacing 20 cm at a temperature of 20 to 25° C. and air humidity below 20% RH. In the process, a nanofiber layer with a weight of 10.75±1.11 g/m², a thickness of 16.94±1.36 μm and a fiber diameter of 231±95 nm was prepared. The prepared nanofiber layer is crosslinked for 60 minutes under UV irradiation at a wavelength of 302 nm. The nanofiber layer thus prepared has an absorption capacity of 2200%/1 hour after full immersion in phosphate buffer and this is also the maximum absorption capacity. The nanofiber structure is maintained for 72 hours and longer, the fusion of the fibers occurs sporadically. This type of material is especially suitable for very exuding wounds.

### Example 4

An electrospinning solution was prepared using a 1:1 mixture of water and isopropyl alcohol as the solvent system. This solution further contained 20 wt. % of the hydrophobized hyaluronic acid derivative (L-HA, Mw 350,000 g/mol, DS 77%) or the pharmaceutically acceptable salt thereof, 75 wt. % of the photocurable ester derivative of hyaluronic acid (F-HA, Mw 98,000 g/mol, DS 5%) or the pharmaceutically acceptable salt thereof and 5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 47.68±1.29 g/m², a thickness of 290±41 μm and a fiber diameter of 214±70 nm was prepared. The prepared nanofiber layer is crosslinked for 60 minutes under UV irradiation at a wavelength of 302 nm. The nanofiber layer thus prepared has an absorption capacity of 1340%/1 hour, the maximum absorption capacity is reached when fully immersed in phosphate buffer (37° C.) in 8 hours and is 1000%. The nanofiber structure is maintained for 72 hours and longer, the fusion of the fibers occurs sporadically. This type of material is especially suitable for very exuding wounds.

### Example 5

An electrospinning solution was prepared using a 1:1 mixture of water and isopropyl alcohol as the solvent system. This solution further contained 48 wt. % of the hydrophobized hyaluronic acid derivative (L-HA, Mw 350,000 g/mol, DS 77%) or the pharmaceutically acceptable salt thereof, 48 wt. % of the photocurable ester derivative of hyaluronic acid (F-HA, Mw 96,000 g/mol, DS 5%) or the pharmaceutically acceptable salt thereof and 4 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 43.31±1.19 g/m², a thickness of 361±73 μm and a fiber diameter of 275±84 nm was prepared. The prepared nanofiber layer is crosslinked for 60 minutes under UV irradiation at a wavelength of 302 nm. The nanofiber layer thus prepared has an absorption capacity of 1300%/1 hour, the maximum absorption capacity is reached when fully immersed in phosphate buffer (37° C.) in 8 hours and is 1320%. The nanofiber structure is maintained for 1 hour, then the fibers are swollen and fused, and after 72 hours a film with slightly preserved pores is formed. This type of material is especially suitable for less exuding wounds.

### Example 6

An electrospinning solution was prepared using a 1:1 mixture of water and isopropyl alcohol as the solvent system. This solution further contained 75 wt. % of the hydrophobized hyaluronic acid derivative (L-HA, Mw 350,000 g/mol, DS 77%) or a pharmaceutically acceptable salt thereof, 20 wt. % of the photocurable ester derivative of hyaluronic acid (F-HA, Mw 98,000 g/mol, DS 5%) or the pharmaceutically acceptable salt thereof and 5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 47.68±3.34 g/m², a thickness of 290±33 μm and a fiber diameter of 235±61 nm was prepared. The prepared nanofiber layer is crosslinked for 150 minutes under UV irradiation at a wavelength of 302 nm. The nanofiber layer thus prepared has an absorption capacity of 1080%/1 hour, the maximum absorption capacity is reached when fully immersed in phosphate buffer (37° C.) in 8 hours and is 1200%. The porous structure is maintained for 72 hours. This type of material is especially suitable for very little exuding wounds.

### Example 7

An electrospinning solution was prepared using a 1:1 mixture of water and isopropyl alcohol as the solvent system. This solution further contained 45.5 wt. % of the hydrophobized hyaluronic acid derivative (L-HA, Mw 350,000 g/mol, DS 77%) or the pharmaceutically acceptable salt thereof, 45.5 wt. % of the photocurable ester derivative of hyaluronic acid (F-HA, Mw 96,000 g/mol, DS 5%) or the pharmaceutically acceptable salt thereof, 5 wt. % native HA and 4 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 4/min, electrode spacing 20 cm at a temperature of 20 to 25° C. and air humidity below 20% RH. In the process, a nanofiber layer with a weight of 8.60±1.89 g/m², a thickness of 12.08±0.51 μm and a fiber diameter of 516±138 nm was prepared. The prepared nanofiber layer is crosslinked for 60 minutes under UV irradiation at a wavelength of 302 nm. The nanofiber layer thus prepared forms a slowly degrading gel upon wetting. The nanofiber layer thus prepared has an absorption capacity of 1980%/1 hour, the maximum absorption capacity is reached when fully immersed in phosphate buffer (37° C.) in 8 hours and is 2730%. The nanofiber structure is maintained for 48 hours. This type of material is especially suitable for less exuding wounds or scars.

### Example 8

An electrospinning solution was prepared using a 1:1 mixture of water and isopropyl alcohol as the solvent system. This solution further contained 70 wt. % of the hydrophobized hyaluronic acid derivative (L-HA, Mw 350,000 g/mol, DS 77%) or the pharmaceutically acceptable salt thereof, 20 wt. % of a photocurable ester derivative of hyaluronic acid (F-HA, Mw 96,000 g/mol, DS 5%) or the pharmaceutically acceptable salt thereof, 5 wt. % native HA and 5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 16.28±1.27 g/m², a thickness of 18.25±1.01 μm and a fiber diameter of 351±102 nm was prepared. The prepared nanofiber layer is crosslinked for 60 minutes under UV irradiation at a wavelength of 302 nm. The nanofiber layer thus prepared forms a very slowly degrading gel after soaking. The nanofiber layer thus prepared has an absorption capacity of 1380%/1 hour, the maximum absorption capacity is reached when fully immersed in phosphate buffer (37° C.) in 8 hours and is 2040%. The nanofiber structure is maintained for 48 hours. This type of material is especially suitable for less exuding wounds or scars.

### Example 9

An electrospinning solution was prepared using a 1:1 mixture of water and isopropyl alcohol as the solvent system. This solution further contained 20 wt. % of the hydrophobized hyaluronic acid derivative (L-HA, Mw 350,000 g/mol, DS 77%) or the pharmaceutically acceptable salt thereof, 70 wt. % of the photo-hardening ester derivative of hyaluronic acid (F-HA, Mw 96,000 g/mol, DS 5%) or a pharmaceutically acceptable salt thereof, 5 wt. % native HA and 5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 57 kV, solution dosing 350 μL/min, electrode spacing 20 cm at a temperature of 20 to 25° C. and air humidity below 20% RH. In the process, a nanofiber layer with a weight of 15.11±1.13 g/m², a thickness of 16.34±0.87 μm and a fiber diameter of 295±81 nm was prepared. The prepared nanofiber layer is crosslinked for 60 minutes under UV irradiation at a wavelength of 302 nm. The nanofiber layer thus prepared forms a very slowly degrading gel after soaking. The nanofiber layer thus prepared has an absorption capacity of 2380%/1 hour, the maximum absorption capacity is reached when fully immersed in phosphate buffer (37° C.) in 8 hours and is 2420%. The nanofiber structure is maintained for 48 hours. This type of material is especially suitable for less exuding wounds or scars.

### Example 10

An electrospinning solution was prepared using a 1:1 mixture of water and isopropyl alcohol as the solvent system. This solution further contained 47.9 wt. % of the hydrophobized hyaluronic acid derivative (L-HA, Mw 350,000 g/mol, DS 77%) or the pharmaceutically acceptable salt thereof, 47.9 wt. % of the photocurable ester derivative of hyaluronic acid (F-HA, Mw 96,000 g/mol, DS 5%) or the pharmaceutically acceptable salt thereof, 4 wt. % polyethylene oxide (Mw=400,000 g/mol) and 0.2 wt. % octenidine. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 6.67±0.38 g/m², a thickness of 8.29±0.28 am and a fiber diameter of 283±106 nm was prepared. The prepared nanofiber layer is crosslinked for 60 minutes under UV irradiation at a wavelength of 302 nm. The nanofiber layer thus prepared has an absorption capacity of 2000%/1 hour, the maximum absorption capacity is reached when fully immersed in phosphate buffer (37° C.) in 8 hours and is 2440%. The nanofiber structure is maintained for 72 hours. This type of material is especially suitable for heavily exuding wounds.

### Example 11

An electrospinning solution was prepared using a 1:1 mixture of water and isopropyl alcohol as the solvent system. This solution further contained 74.8 wt. % of a hydrophobized hyaluronic acid derivative (L-HA, Mw 350,000 g/mol, DS 77%) or a pharmaceutically acceptable salt thereof, 20 wt. % of a photo-hardening ester derivative of hyaluronic acid (F-HA, Mw 96,000 g/mol, DS 5%) or a pharmaceutically acceptable salt thereof, 5 wt. % polyethylene oxide (Mw=400,000 g/mol) and 0.2 wt. % octenidine. The total dry matter concentration in the solution is 3 wt. %. The weight % of the individual components mentioned above is based on the dry matter in the spinning solution. The solution was electrostatically spun by a moving needle-free nozzle on a rotating collector 25 cm wide at a voltage of 56 kV, solution dosing 350 μL/min, electrode spacing 20 cm at a temperature of 20 to 25° C. and air humidity below 20% RH. In the process, a nanofiber layer with a weight of 14.70±0.82 g/m², a thickness of 16.12±0.17 μm and a fiber diameter of 286±94 nm was prepared. The prepared nanofiber layer is crosslinked for 60 minutes under UV irradiation at a wavelength of 302 nm. The nanofiber layer thus prepared has an absorption capacity of 1230%/1 hour, the maximum absorption capacity is reached when fully immersed in phosphate buffer (37° C.) in 8 hours and is 1350%. The nanofiber structure is maintained for 48 hours. This type of material is especially suitable for weakly exuding wounds.

### Example 12

An electrospinning solution was prepared using a 1:1 mixture of water and isopropyl alcohol as the solvent system. This solution further contained 20 wt. % of the hydrophobized hyaluronic acid derivative (L-HA, Mw 350,000 g/mol, DS 77%) or the pharmaceutically acceptable salt thereof, 74.8 wt. % of the photocurable ester derivative of hyaluronic acid (F-HA, Mw 96,000 g/mol, DS 5%) or the pharmaceutically acceptable salt thereof, 5 wt. % polyethylene oxide (Mw=400,000 g/mol) and 0.2 wt. % octenidine. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 56 kV, solution dosing 350 μL/min, electrode spacing 20 cm at a temperature of 20 to 25° C. and air humidity below 20% RH. In the process, a nanofiber layer with a weight of 12.87±0.16 g/m², a thickness of 13.78±1.01 μm and a fiber diameter of 307±115 nm was prepared. The prepared nanofiber layer is crosslinked for 60 minutes under UV irradiation at a wavelength of 302 nm. The nanofiber layer thus prepared has an absorption capacity of 2040%/1 hour, the maximum absorption capacity is reached when fully immersed in phosphate buffer (37° C.) in 8 hours and is 2510%. The nanofiber structure is maintained for 72 hours and longer. This type of material is especially suitable for heavily exuding wounds.

### Example 13

An electrospinning solution was prepared using a 1:1 mixture of water and isopropyl alcohol as the solvent system. This solution further contained 47 wt. % of the hydrophobized hyaluronic acid derivative (L-HA, Mw 350,000 g/mol, DS 77%) or the pharmaceutically acceptable salt thereof of formula II, 47 wt. % of the photocurable ester derivative of hyaluronic acid (F-HA, Mw 96,000 g/mol, DS 5%) or the pharmaceutically acceptable salt thereof, 4 wt. % polyethylene oxide (Mw=400,000 g/mol) and 2 wt. % salicylic acid. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 6.02±0.34 g/m², a thickness of 7.38±0.39 μm and a fiber diameter of 402±150 nm was prepared. The prepared nanofiber layer is crosslinked for 60 minutes under UV irradiation at a wavelength of 302 nm. The nanofiber layer thus prepared has an absorption capacity of 2700%/1 hour, the maximum absorption capacity is reached by full immersion in phosphate buffer (37° C.) in 1 hour. The nanofiber structure is maintained for 1 hour, then the fibers are swollen and fused, and after 3 hours a film with slightly preserved pores is formed. This type of material is especially suitable for minimally exuding wounds.

### Example 14

An electrospinning solution was prepared using a 1:1 mixture of water and isopropyl alcohol as the solvent system. This solution further contained 73 wt. % of the hydrophobized hyaluronic acid derivative (L-HA, Mw 350,000 g/mol, DS 77%) or the pharmaceutically acceptable salt thereof of formula II, 20 wt. % of the photocurable ester derivative of hyaluronic acid (F-HA, Mw 96,000 g/mol, DS 5%) or the pharmaceutically acceptable salt thereof, 5 wt. % polyethylene oxide (Mw=400,000 g/mol) and 2 wt. % salicylic acid. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 56 kV, solution dosing 350 μL/min, electrode spacing 20 cm at a temperature of 20 to 25° C. and air humidity below 20% RH. In the process, a nanofiber layer with a weight of 12.54±0.18 g/m², a thickness of 14.07±0.93 μm and a fiber diameter of 304±112 nm was prepared. The prepared nanofiber layer is crosslinked for 60 minutes under UV irradiation at a wavelength of 302 nm. The nanofiber layer thus prepared has an absorption capacity of 1603%/1 hour, the maximum absorption capacity is reached by full immersion in phosphate buffer (37° C.) in 1 hour. The nanofiber structure is maintained for 1 hour, then the fibers are swollen and fused, and after 3 hours a film with slightly preserved pores is formed. This type of material is especially suitable for minimally exuding wounds.

### Example 15

An electrospinning solution was prepared using a 1:1 mixture of water and isopropyl alcohol as the solvent system. This solution further contained 20 wt. % of the hydrophobized hyaluronic acid derivative (L-HA, Mw 350,000 g/mol, DS 77%) or the pharmaceutically acceptable salt thereof of formula II, 73 wt. % of the photocurable ester derivative of hyaluronic acid (F-HA, Mw 96,000 g/mol, DS 5%) or the pharmaceutically acceptable salt thereof, 5 wt. % polyethylene oxide (Mw=400,000 g/mol) and 2 wt. % salicylic acid. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 56 kV, solution dosing 350 μL/min, electrode spacing 20 cm at a temperature of 20 to 25° C. and air humidity below 20% RH. In the process, a nanofiber layer with a weight of 10.48±0.28 g/m², a thickness of 11.07±1.16 μm and a fiber diameter of 208±106 nm was prepared. The prepared nanofiber layer is crosslinked for 60 minutes under UV irradiation at a wavelength of 302 nm. The nanofiber layer thus prepared has an absorption capacity of 2540%/1 hour, the maximum absorption capacity is reached by full immersion in phosphate buffer (37° C.) in 1 hour. The nanofiber structure is maintained for 8 hours, followed by swelling and partial fusion of the fibers. This type of material is especially suitable for more exuding wounds.

### Example 16

An electrospinning solution was prepared using a 1:1 mixture of water and isopropyl alcohol as the solvent system. This solution further contained 45.5 wt. % of the hydrophobized hyaluronic acid derivative (L-HA, Mw 350,000 g/mol, DS 77%) or the pharmaceutically acceptable salt thereof of formula II, 45.5 wt. % of the photocurable ester derivative of hyaluronic acid (F-HA, Mw 96,000 g/mol, DS 5%) or the pharmaceutically acceptable salt thereof of formula I, 4 wt. % polyethylene oxide (Mw=400,000 g/mol) and 5 wt. % triclosan. The total dry matter concentration in the solution is 3 wt. %. The weight % of the individual components mentioned above is based on the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of to 25° C. and air humidity below 20% RH. In the process, a nanofiber layer with a weight of 9.36±0.20 g/m², a thickness of 13.76±1.20 μm and a fiber diameter of 243±44 nm was prepared. The prepared nanofiber layer is crosslinked for 60 minutes under UV irradiation at a wavelength of 302 nm. The nanofiber layer thus prepared has an absorption capacity of 1730%/1 hour, the maximum absorption capacity is reached when fully immersed in phosphate buffer (37° C.) in 8 hours and is 1830%. The nanofiber structure is maintained for 48 hours, then the fibers are swollen and fused, and after 72 hours a film with preserved pores is formed. This type of material is especially suitable for heavily exuding wounds.

### Example 17

An electrospinning solution was prepared using a 1:1 mixture of water and isopropyl alcohol as the solvent system. This solution further contained 70 wt. % of the hydrophobized hyaluronic acid derivative (L-HA, Mw 350,000 g/mol, DS 77%) or the pharmaceutically acceptable salt thereof of formula II, 20 wt. % of the photocurable ester derivative of hyaluronic acid (F-HA, Mw 96,000 g/mol, DS 5%) or the pharmaceutically acceptable salt thereof of formula I, 5 wt. % polyethylene oxide (Mw=400,000 g/mol) and 5 wt. % triclosan. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 17.22±0.45 g/m², a thickness of 18.06±0.54 μm and a fiber diameter of 375±71 nm was prepared. The prepared nanofiber layer is crosslinked for 60 minutes under UV irradiation at a wavelength of 302 nm. The nanofiber layer thus prepared has an absorption capacity of 1360%/1 hour, the maximum absorption capacity is reached when fully immersed in phosphate buffer (37° C.) in 8 hours and is 1650%. The nanofiber structure is maintained for 48 hours, then the fibers are swollen and fused, and after 72 hours a film with preserved pores is formed. This type of material is especially suitable for weakly exuding wounds.

### Example 18

An electrospinning solution was prepared using a 1:1 mixture of water and isopropyl alcohol as the solvent system. This solution further contained 20 wt. % of the hydrophobized hyaluronic acid derivative (L-HA, Mw 350,000 g/mol, DS 77%) or the pharmaceutically acceptable salt thereof of formula II, 70 wt. % of the photocurable ester derivative of hyaluronic acid (F-HA, Mw 96,000 g/mol, DS 5%) or the pharmaceutically acceptable salt thereof of formula I, 5 wt. % polyethylene oxide (Mw=400,000 g/mol) and 5 wt. % triclosan. The total dry matter concentration in the solution is 3 wt. %. The weight % of the individual components mentioned above is relative to the dry matter in the spinning solution. The solution was electrostatically spun by a moving needle-free nozzle on a rotating collector 25 cm wide at a voltage of 55 kV, solution dosing 350 μL/min, electrode spacing 20 cm at a temperature of 20 to 25° C. and air humidity below 20% RH. In the process, a nanofiber layer with a weight of 14.72±0.48 g/m², a thickness of 16.89±0.77 μm and a fiber diameter of 235±105 nm was prepared. The prepared nanofiber layer is crosslinked for 60 minutes under UV irradiation at a wavelength of 302 nm. The nanofiber layer thus prepared has an absorption capacity of 2120%/1 hour, the maximum absorption capacity is reached when fully immersed in phosphate buffer (37° C.) in 8 hours and is 2308%. The nanofiber structure is maintained for 72 hours and longer. This type of material is especially suitable for heavily exuding wounds.

### Example 19

An electrospinning solution was prepared using a 1:1 mixture of water and isopropyl alcohol as the solvent system. This solution further contained 75 wt. % of the hydrophobized hyaluronic acid derivative (L-HA, Mw 320,000 g/mol, DS 73%) or the pharmaceutically acceptable salt thereof, 21.5 wt. % of the photocurable ester derivative of hyaluronic acid (F-HA, Mw 98,000 g/mol, DS 5%) or the pharmaceutically acceptable salt thereof and 3.5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 55 kV, solution dosing 350 μL/min, electrode spacing 20 cm at a temperature of 20 to 25° C. and air humidity below 20% RH. In the process, a nanofiber layer with a weight of 8.12±0.21 g/m², a thickness of 11.03±1.16 μm and a fiber diameter of 351±102 nm was prepared. The prepared nanofiber layer is crosslinked for 60 minutes under UV irradiation at a wavelength of 302 nm. The nanofiber layer thus prepared has an absorption capacity of 1230%/1 hour, the maximum absorption capacity is reached when fully immersed in phosphate buffer (37° C.) in 8 hours and is 1480%. The nanofiber structure is maintained for 1 hour, then the fibers are swollen and fused, and after 72 hours a film with slightly preserved pores is formed. This type of material is especially suitable for less exuding wounds.

### Example 20

An electrospinning solution was prepared using a 1:1 mixture of water and isopropyl alcohol as the solvent system. This solution further contained 48.5 wt. % of the hydrophobized hyaluronic acid derivative (L-HA, Mw 320,000 g/mol, DS 73%) or the pharmaceutically acceptable salt thereof, 48 wt. % of the photocurable ester derivative of hyaluronic acid (F-HA, Mw 98,000 g/mol, DS 5%) or the pharmaceutically acceptable salt thereof and 3.5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 55 kV, solution dosing 350 μL/min, electrode spacing 20 cm at a temperature of 20 to 25° C. and air humidity below 20% RH. In the process, a nanofiber layer with a weight of 9.29±0.43 g/m², a thickness of 12.05±0.19 μm and a fiber diameter of 460±103 nm was prepared. The prepared nanofiber layer is crosslinked for 60 minutes under UV irradiation at a wavelength of 302 nm. The nanofiber layer thus prepared has an absorption capacity of 1200%/1 hour, the maximum absorption capacity is reached when fully immersed in phosphate buffer (37° C.) in 8 hours and is 2300%. The nanofiber structure is maintained for 48 hours, followed by swelling and fusion of the fibers and enlarging of the pores. This type of material is especially suitable for more exuding wounds.

### Example 21

An electrospinning solution was prepared using a 1:1 mixture of water and isopropyl alcohol as the solvent system. This solution further contained 21.5 wt. % of the hydrophobized hyaluronic acid derivative (L-HA, Mw 320,000 g/mol, DS 73%) or the pharmaceutically acceptable salt thereof, 75 wt. % of the photocurable ester derivative of hyaluronic acid (F-HA, Mw 98,000 g/mol, DS 5%) or the pharmaceutically acceptable salt thereof and 3.5 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 56 kV, solution dosing 350 μL/min, electrode spacing 20 cm at a temperature of 20 to 25° C. and air humidity below 20% RH. In the process, a nanofiber layer with a weight of 11.01±2.17 g/m², a thickness of 13.73±1.42 μm and a fiber diameter of 262±86 nm was prepared. The prepared nanofiber layer is crosslinked for 60 minutes under UV radiation at a wavelength of 302 nm. The nanofiber layer thus prepared has an absorption capacity of 2400%/1 hour after full immersion in phosphate buffer and is also the maximum absorption capacity. The nanofiber structure is maintained for 72 hours and longer, the fusion of the fibers occurs sporadically. This type of material is especially suitable for very exuding wounds.

### Example 22

An electrospinning solution was prepared using a 1:1 mixture of water and isopropyl alcohol as the solvent system. This solution further contained 75 wt. % of the hydrophobized hyaluronic acid derivative (L-HA, Mw 320,000 g/mol, DS 73%) or the pharmaceutically acceptable salt thereof, 15 wt. % of the photocurable ester derivative of hyaluronic acid (F-HA, Mw 98,000 g/mol, DS 5%) or the pharmaceutically acceptable salt thereof and 10 wt. % polyethylene oxide (Mw=400,000 g/mol). The total dry matter concentration in the solution is 3 wt. %. The weight % of the individual components mentioned above is based on the dry matter in the spinning solution. The solution was electrostatically spun with a needle-free nozzle on a rotating collector 10 cm wide at a voltage of 54 kV, solution dosing 350 μL/min, electrode spacing 20 cm at a temperature of 20 to 25° C. and air humidity below 20% RH. In the process, a nanofiber layer with a weight of 9.20±1.37 g/m², a thickness of 12.96±2.13 μm and a fiber diameter of 334±95 nm was prepared. The prepared nanofiber layer is crosslinked for 60 minutes under UV irradiation at a wavelength of 302 nm. The nanofiber layer thus prepared has an absorption capacity of 1250%/1 hour, the maximum absorption capacity is reached when fully immersed in phosphate buffer (37° C.) in 8 hours and is 1630%. The nanofiber structure is maintained for 3 hours, then the fibers are swollen and fused, and after 72 hours a film with slightly preserved pores is formed. This type of material is especially suitable for less exuding wounds.

### Example 23

An electrospinning solution was prepared using a 1:1 mixture of water and isopropyl alcohol as the solvent system. This solution further contained 45 wt. % of the hydrophobized hyaluronic acid derivative (L-HA, Mw 320,000 g/mol, DS 73%) or the pharmaceutically acceptable salt thereof, 45 wt. % of the photocurable ester derivative of hyaluronic acid (F-HA, Mw 98,000 g/mol, DS 5%) or the pharmaceutically acceptable salt thereof and 10 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 54 kV, solution dosing 350 4/min, electrode spacing 20 cm at a temperature of 20 to 25° C. and air humidity below 20% RH. In the process, a nanofiber layer with a weight of 7.42±0.71 g/m², a thickness of 8.95±0.16 μm and a fiber diameter of 437±135 nm was prepared. The prepared nanofiber layer is crosslinked for 60 minutes under UV irradiation at a wavelength of 302 nm. The nanofiber layer thus prepared has an absorption capacity of 1540%/1 hour, the maximum absorption capacity is reached when fully immersed in phosphate buffer (37° C.) in 8 hours and is 2200%. The nanofiber structure is maintained for 48 hours, followed by swelling and fusion of the fibers and enlarging of the pores. This type of material is especially suitable for more exuding wounds.

### Example 24

An electrospinning solution was prepared using a 1:1 mixture of water and isopropyl alcohol as the solvent system. This solution further contained 15 wt. % of the hydrophobized hyaluronic acid derivative (L-HA, Mw 320,000 g/mol, DS 73%) or the pharmaceutically acceptable salt thereof, 75 wt. % of the photocurable ester derivative of hyaluronic acid (F-HA, Mw 98,000 g/mol, DS 5%) or the pharmaceutically acceptable salt thereof and 10 wt. % polyethylene oxide (Mw=400,000 g/mol). The total dry matter concentration in the solution is 3 wt. %. The weight % of the individual components mentioned above is relative to the dry matter in the spinning solution. The solution was electrostatically spun with a needle-free nozzle on a rotating collector 10 cm wide at a voltage of 56 kV, solution dosing 350 4/min, electrode spacing 20 cm at a temperature of 20 to 25° C. and air humidity below 20% RH. In the process, a nanofiber layer with a weight of 9.64±1.07 g/m², a thickness of 9.17±0.36 μm and a fiber diameter of 249±102 nm was prepared. The prepared nanofiber layer is crosslinked for 60 minutes under UV radiation at a wavelength of 302 nm. The nanofiber layer thus prepared has an absorption capacity of 2280%/1 hour after full immersion in phosphate buffer and is also the maximum absorption capacity. The nanofiber structure is maintained for 72 hours and longer, the fusion of the fibers occurs sporadically. This type of material is especially suitable for very exuding wounds.

### Example 25

The nanofiber layers were prepared according to Examples 1 to 24, using an absorbent layer of synthetic or natural cellulose fleece or polyester as the substrate to which they were applied.

### Example 26

The nanofiber layers were prepared according to Examples 1 to 24, using a waterproof porous polyethylene film as the substrate to which they were applied.

### Example 27

Nanofiber layers were prepared according to Examples 1, 2 and 3 and were photocured for 50 and 90 minutes.

### Example 28

The nanofiber layers were prepared according to Examples 1 to 9, using a 2:3 mixture of water and isopropyl alcohol as the solvent system.

### Example 29

TABLE 1
Summary of the parameters* of nanofibrous layers prepared according to the Examples above.

| Relative | Substit. | | | | | | | | | | |
|---|---|---|---|---|---|---|---|---|---|---|---|
| proportion of | degree | | | | Sol. | Spin- | UV | Absorpt. | Layer | | |
| components in the | L-HA/ | Mw | | | system | ning | irrad. | capacity/ | thick- | | Fiber |
| dry matter/fiber | F-HA | L-HA/F-HA | Mw PEO | Concent. | IPA/water | time | time | 1 h | ness | Grammage | diammeter |
| layer [wt. %] | [%] | [kg/mol] | [kg/mol] | [wt. %] | [wt. %] | [min] | [min] | [%] | [μm] | [g/m2] | [nm] |
| L-HA/F-HA/PEO | 65/5.13 | 320/96 | 400 | 3 | 70/30 | 50 | 60 | 1312 | 6.26 | 7.23 | 312 |
| 75/20/5 | 65/5.13 | 320/96 | 400 | 3 | 60/40 | 50 | 60 | 1060 | 14.26 | 10.23 | 420 |
| | 65/5.13 | 320/96 | 400 | 3 | 60/40 | 60 | 60 | 1230 | 16.93 | 11.25 | 390 |
| | 65/5.4 | 320/98 | 400 | 3 | 50/50 | 80 | 60 | 1670 | 16.79 | 12.67 | 231 |
| | 65/5.4 | 320/98 | 600 | 3 | 50/50 | 60 | 60 | 1420 | 13.42 | 14.75 | 405 |
| | 73/5.15 | 320/86 | 400 | 3 | 50/50 | 60 | 60 | 1280 | 12.05 | 11.41 | 462 |
| | 73/5.4 | 320/98 | 400 | 3 | 50/50 | 60 | 90 | 1230 | 11.97 | 11.03 | 377 |
| | 77/5.13 | 350/96 | 400 | 3 | 50/50 | 150 | 60 | 1080 | 290.03 | 47.68 | 235 |
| | 89/5.4 | 340/98 | 400 | 3 | 50/50 | 60 | 60 | 1140 | 13.07 | 12.06 | 457 |
| | 73/5.4 | 320/98 | 400 | 3 | 50/50 | 60 | 50 | 1000 | 15.77 | 11.11 | 304 |
| L-HA/F-HA/PEO | 73/5.4 | 320/98 | 400 | 3 | 50/50 | 60 | 60 | 1230 | 11.03 | 8.12 | 351 |
| 75/21.5/3.5 | | | | | | | | | | | |
| L-HA/F-HA/PEO | 73/5.4 | 320/98 | 400 | 3 | 50/50 | 60 | 60 | 1250 | 12.96 | 9.20 | 334 |
| 75/15/10 | | | | | | | | | | | |
| L-HA/F-HA/HA/PEO | 77/5.13 | 350/96 | 400 | 3 | 50/50 | 60 | 60 | 1380 | 18.25 | 16.28 | 351 |
| 70/20/5/5 | | | | | | | | | | | |
| L-HA/F- | 77/5.13 | 350/96 | 400 | 3 | 50/50 | 60 | 60 | 1230 | 16.12 | 14.70 | 286 |
| HA/OCT/PEO | | | | | | | | | | | |
| 74.8/20/0.2/5 | | | | | | | | | | | |
| L-HA/F-HA/KS/PEO | 77/5.13 | 350/96 | 400 | 3 | 50/50 | 60 | 60 | 1603 | 14.07 | 12.54 | 304 |
| 73/20/2/5 | | | | | | | | | | | |
| L-HA/F-HA/TRI/PEO | 77/5.13 | 350/96 | 400 | 3 | 50/50 | 60 | 60 | 1360 | 18.06 | 17.22 | 375 |
| 70/20/5/5 | | | | | | | | | | | |

| Relative | Substit. | | | | | | | | | | |
|---|---|---|---|---|---|---|---|---|---|---|---|
| proportion of | degree | | | | Sol. | Spin- | UV | Absorpt. | Layer | | |
| components in the | L-HA/ | Mw | | | system | ning | irrad. | capacity/ | thick- | | Fiber |
| dry matter/fiber | F-HA | L-HA/F-HA | Mw PEO | Concent. | IPA/water | time | time | 1 h | ness | Grammage | diammeter |
| layer [wt. %] | [%] | [kg/mol] | [kg/mol] | [wt. %] | [wt. %] | [min] | [min] | [%] | [μm] | [g/m²] | [nm] |
| L-HA/F-HA/PEO | 65/5.13 | 320/96 | 400 | 4 | 50/50 | 50 | 60 | 1700 | 13.76 | 7.93 | 394 |
| 48/48/4 | 65/5.13 | 320/96 | 400 | 4 | 60/40 | 50 | 60 | 1840 | 14.78 | 9.03 | 501 |
| | 65/5.4 | 320/98 | 400 | 4 | 50/50 | 60 | 60 | 1500 | 11.66 | 12.51 | 402 |
| | 65/5.4 | 320/98 | 400 | 3 | 50/50 | 80 | 60 | 2310 | 15.20 | 16.47 | 372 |
| | 65/5.4 | 320/98 | 600 | 3 | 50/50 | 60 | 60 | 2240 | 14.02 | 14.78 | 431 |
| | 89/5.4 | 340/98 | 400 | 3 | 50/50 | 60 | 60 | 1980 | 16.24 | 15.87 | 287 |
| | 73/5.15 | 320/86 | 400 | 3 | 50/50 | 60 | 90 | 2150 | 17.21 | 16.32 | 402 |
| | 73/5.15 | 320/86 | 400 | 3 | 50/50 | 60 | 50 | 2520 | 14.64 | 12.12 | 479 |
| | 73/5.4 | 320/98 | 400 | 3 | 50/50 | 60 | 60 | 2000 | 11.75 | 7.29 | 356 |
| | 77/5.4 | 350/96 | 400 | 3 | 50/50 | 150 | 60 | 1300 | 361.12 | 43.31 | 275 |
| L-HA/F-HA/PEO | 73/5.4 | 320/98 | 400 | 3 | 50/50 | 60 | 60 | 1200 | 12.05 | 9.29 | 460 |
| 48.5/48/3.5 | | | | | | | | | | | |
| L-HA/F-HA/PEO | 73/5.4 | 320/98 | 400 | 3 | 50/50 | 60 | 60 | 1540 | 8.95 | 7.42 | 457 |
| 45/45/10 | | | | | | | | | | | |
| L-HA/F- | 77/5.13 | 350/96 | 400 | 3 | 50/50 | 60 | 60 | 2200 | 18.14 | 17.45 | 516 |
| HA/HA/PEO | | | | | | | | | | | |
| 45.5/45.5/5/4 | | | | | | | | | | | |
| L-HA/F- | 77/5.13 | 350/96 | 400 | 3 | 50/50 | 60 | 60 | 2440 | 6.67 | 8.29 | 283 |
| HA/OCT/PEO | | | | | | | | | | | |
| 47.9/47.9/0.2/4 | | | | | | | | | | | |
| L-HA/F- | 77/5.13 | 350/96 | 400 | 3 | 50/50 | 60 | 60 | 2700 | 6.04 | 7.38 | 402 |
| HA/KS/PEO | | | | | | | | | | | |
| 47/47/2/4 | | | | | | | | | | | |
| L-HA/F- | 77/5.13 | 350/96 | 400 | 3 | 50/50 | 60 | 60 | 1730 | 13.76 | 9.36 | 243 |
| HA/TRI/PEO | | | | | | | | | | | |
| 45.5/45.5/5/4 | | | | | | | | | | | |
| L-HA/F-HA/PEO | 65/5.13 | 320/96 | 400 | 4 | 50/50 | 50 | 60 | 1940 | 16.12 | 8.52 | 201 |
| 20/75/5 | 65/5.13 | 320/96 | 400 | 4 | 60/40 | 50 | 60 | 2100 | 14.02 | 7.45 | 290 |
| | 65/5.15 | 320/86 | 400 | 4 | 50/50 | 60 | 60 | 2150 | 17.20 | 9.23 | 266 |
| | 89/5.13 | 340/96 | 400 | 3 | 50/50 | 60 | 60 | 1630 | 20.26 | 13.44 | 224 |
| | 73/5.4 | 320/98 | 400 | 3 | 50/50 | 60 | 60 | 2200 | 16.95 | 10.81 | 269 |
| | 73/5.4 | 320/98 | 600 | 3 | 50/50 | 60 | 60 | 3480 | 16.58 | 14.01 | 335 |
| | 73/5.4 | 320/98 | 400 | 3 | 50/50 | 80 | 50 | 3220 | 18.20 | 16.72 | 301 |
| | 73/5.4 | 320/98 | 400 | 3 | 50/50 | 60 | 90 | 1810 | 16.34 | 14.28 | 247 |
| | 77/513 | 350/96 | 400 | 3 | 50/50 | 150 | 60 | 1240 | 237.25 | 39.18 | 214 |
| L-HA/F-HA/PEO | 73/5.4 | 320/98 | 400 | 3 | 50/50 | 60 | 60 | 2400 | 13.73 | 11.01 | 262 |
| 21.5/75/3.5 | | | | | | | | | | | |
| L-HA/F-HA/PEO | 73/5.4 | 320/98 | 400 | 3 | 50/50 | 60 | 60 | 2280 | 9.17 | 9.64 | 249 |
| 15/75/10 | | | | | | | | | | | |
| L-HA/F- | 77/5.13 | 350/96 | 400 | 3 | 50/50 | 60 | 60 | 2380 | 16.34 | 15.11 | 295 |
| HA/HA/PEO | | | | | | | | | | | |
| 20/70/5/5 | | | | | | | | | | | |
| L-HA/F- | 77/5.13 | 350/96 | 400 | 3 | 50/50 | 60 | 60 | 2040 | 13.78 | 12.87 | 307 |
| HA/OCT/PEO | | | | | | | | | | | |
| 20/74.8/0.2/5 | | | | | | | | | | | |
| L-HA/F- | 77/5.13 | 350/96 | 400 | 3 | 50/50 | 60 | 60 | 2540 | 11.07 | 10.48 | 208 |
| HA/SA/PEO | | | | | | | | | | | |
| 20/73/2/5 | | | | | | | | | | | |
| L-HA/F- | 77/5.13 | 350/96 | 400 | 3 | 50/50 | 60 | 60 | 2120 | 16.89 | 14.72 | 235 |
| HA/TRI/PEO | | | | | | | | | | | |
| 20/70/0/5 | | | | | | | | | | | |

*nanofibrous layers were spun alternately to the base of polyethylene foil, natural or synthetic cellulose fleece and polyester.
OCT (octenidine), SA (salicylic acid) and TRI (triclosan)

### Example 30

TABLE 2
Summary of parameters* of nanofibrous layers prepared according to Examples 1, 2 and 3**; see above

| Relative | | | | | | | | | | | |
|---|---|---|---|---|---|---|---|---|---|---|---|
| proportion of | Substitution | | | | | | | | | | |
| components | degree | | | | Sol. | | UV | Absorpt. | | | |
| in the dry | L-HA/ | Mw | Mw | | system | Spinning | irrad. | capacity/ | | | Fiber |
| matter/fiber | F-HA | L-HA/F-HA | PEO | Concentr. | IPA/water | time | time | 1 h | Thickness | Grammage | diammeter |
| layer [wt. %] | [%] | [kg/mol] | [kg/mol] | [wt. %] | [wt. %] | [min] | [min] | [%] | [μm] | [g/m²] | [nm] |
| L-HA/F- | 73/4.2 | 320/82 | 400 | 3 | 50/50 | 60 | 60 | 1500-2150 | 7.14 | 8.14 | 218 |
| HA/PEO | 73/17 | 320/100 | 400 | 3 | 50/50 | 60 | 60 | | 5.47 | 5.48 | 230 |
| 20/75/5 | 73/20 | 320/110 | 400 | 3 | 50/50 | 60 | 60 | | 6.52 | 6.87 | 261 |
| | 73/10.8 | 320/97 | 400 | 3 | 50/50 | 60 | 60 | | 6.12 | 7.30 | 259 |
| L-HA/F- | 94/5.13 | 310/96 | 400 | 3 | 50/50 | 60 | 60 | 1100-1280 | 9.73 | 13.05 | 405 |
| HA/PEO | 94/5.4 | 310/98 | 400 | 3 | 50/50 | 60 | 60 | | 10.12 | 12.49 | 398 |
| 75/20/5 | 94/10 | 310/97 | 400 | 3 | 50/50 | 60 | 60 | | 10.08 | 10.71 | 374 |
| | 73/4.2 | 320/82 | 400 | 3 | 50/50 | 60 | 60 | | 9.45 | 9.81 | 362 |
| L-HA/F- | 89/5.13 | 340/96 | 300 | 5 | 50/50 | 60 | 60 | 2270 | 10.65 | 11.21 | 317 |
| HA/PEO | | | | | | | | | | | |
| 48/48/4 | | | | | | | | | | | |
| L-HA/F- | 65/5.13 | 320/96 | 900 | 2 | 50/50 | 60 | 60 | 1050 | 13.05 | 15.84 | 460 |
| HA/PEO | | | | | | | | | | | |
| 20/75/5 | | | | | | | | | | | |

*nanofibrous layers were spun alternately to the base of polyethylene foil, natural o synthetic fleece and polyester.
**for the preparation of nanofibrous layers from Table 2, identical technological processes stated in Examples 1, 2 and 3 were used.

### REFERENCES

1) HASATSRI, Sukhontha, et al. Comparison of the morphological and physical properties of different absorbent wound dressings. Dermatology research and practice. 2018, 2018.
2) R. S. Ambekar, B. Kandasubramanian. Advancements in nanofihers for wound dressing: A review European Polymer Journal. 117 (2019) 304-336. https://doi.org/10.1016/j.eurpolymj.2019.05.020.
3) S. Homaeigohar, A. R. Boccaccini, Antibacterial hiohybrid nanofibers for wound dressings, Acta Biomaterialia, 107 (2020) 25-49. https://doi.or2110,101.6/j.actbio.2020.02.022,
4) Y. Liu, S. Zhou, Y, Ciao, Y. Zhai, Electrospun nanofibers as a wound dressing for treating diabetic foot ulcer. Asian Journal of Pharmaceutical Sciences. 14 (2019) 130-143. https://doi.org/10.1016/j.ajps.2018.04.004.
5) K. M. Woo, V. J. Chen, P. X. Ma, Nano-fibrous scaffolding architecture selectively enhances protein adsorption contributing to cell attachment, J. Biomed. Mater. Res. 67A (2003) 531-537, https://doi.org/10.1002/jbm.a. 10098,
6) V. Jayarama Reddy, S. Radhakrishnan, R. Ravichandran, S. Mukherjee, R. Balainurugan, S. Sundarrajan, S. Ramakrishna, Nanofibrous structured biomimetic strategies for skin tissue regeneration: Nanofibrous structures for wound healing, Wound Repair Regen. 21 (2013) 1-16. https://doi.org/10.1111/j.1524-475X.2012.00861.x.
7) GHASEMI-MOBARAKEH, Laleh, et al, Electrospun poly (ε-caprolactone)/gelatin nanofibrous scaffolds for nerve tissue engineering. Biomaterials, 2008, 29.34: 4532-4539.
8) VÍTKOVÁ, Lenka, et al. Electrospinning of hyaluronan using polymer coelectrospinning and intermediate solvent. Polymers, 2019, 11.9: 1517.
9) AHIRE, J. J., et al, Polyethylene oxide (PEO)-hyaluronic acid (HA) nanofihers with kanamycin inhibits the growth of Listeria monocytogenes. Biomedicine Pharmacotherapy, 2017, 86: 143-148.
10) CHEN, Guangkai, et al. Preparation, characterization, and application of PEO/HA core shell nanofihers based on electric field induced phase separation during electrospinning Polymer, 2016, 83: 12-19.
11) SINGH, Baljeet, et al. Development, optimization, and characterization of polymeric electrospun nanofiber: a new attempt in sublingual delivery of nicorandil for the management of angina pectoris. Artificial cells, nanomedicine, and biotechnology, 2016, 44.6: 1498-1507.
12) FIGUEIRA, Daniela R., et al. Production and characterization of polycaprolactone-hyaluronic acid/chitosan-zein electrospun bilayer nanofibrous membrane for tissue regeneration. International journal of biological macromolecules, 2016, 93: 1100-1110,
13) ENTEKHABI, Elahe, et al, Design and manufacture of neural tissue engineering scaffolds using hyaluronic acid and polycaprolactone nanofibers with controlled porosity. Materials Science and Engineering: C, 2016, 69: 380-387.
14) WANG, Zhenhei, et al. Evaluation of emulsion electrospun polycaprolactone/hyaluronan/epidermal growth factor nanofibrous scaffolds for wound healing. Journal of biomaterials applications, 2016, 30.6: 686-698.
15) LIU, Yang, et al. Effects of solution properties and electric field on the electrospinning of hyaluronic acid. Carbohydrate Polymers, 2011, 83.2: 1011-1015.
16) UM, In Chul, et al. Electro-spinning and electro-blowing of hyaluronic acid. Biomacromolecules, 2004, 5.4: 1428-1436.
17) PABJAŃCZYK-WLAZLO, E., et al. Fabrication of Pure Electrospun Materials from Hyaluronic Acid. Fibres & Textiles in Eastern Europe, 2017.
18) XUE, Yu, et al. Synthesis of hyaluronic acid hydrogels by crosslinking the mixture of high-molecular-weight hyaluronic acid and low-molecular-weight hyaluronic acid with 1,4-butanediol diglycidyl ether. RSC Advances, 2020, 10.12: 7206-7213.
19) YEOM, Junseok, et al. Effect of cross-linking reagents for hyaluronic acid hydrogel dermal fillers on tissue augmentation and regeneration. Bioconjugate chemistry, 2010, 21.2: 240-247.
20) JI, Yuan, et al. Dual-syringe reactive electrospinning of cross-linked hyaluronic acid hydrogel nanofibers for tissue engineering applications. Macromolecular bioscience, 2006, 6.10: 811-817.
21) KIM. Iris L., et al. Fibrous hyaluronic acid hydrogels that direct MSC chondrogenesis through mechanical and adhesive cues. Biomaterials, 2013, 34.22: 5571-5580,
22) HUERTA-ANGELES, Gloria, et al. Synthesis of photo-crosslinkable hyaluronan with tailored degree of substitution suitable for production of water resistant nanofibers. Carbohydrate polymers, 2016, 137: 255-263.
23) LU, Hangyi, et al. Electrospun water-stable rein/ethyl cellulose composite nanofiber and its drug release properties. Materials Science and Engineering: C, 2017, 74: 86-93,
24) LUBASOVA, Daniela; MULLEROVA, Jana; NETRAVALI, Anil N. Water-resistant plant protein-based nanofiber membranes. Journal of Applied Polymer Science, 2015, 132.16.
25) MOVAHEDI, Mehdi, et al. Potential of novel electrospun core-shell structured polyurethane/starch (hyaluronic acid) nanofibers for skin tissue engineering: In vitro and in vivo evaluation. International Journal of Biological Macromolecules, 2020, 146: 627-637.
26) ESKANDARINIA, Asghar, et al. A propolis enriched polyurethane-hyaluronic acid nanofibrous wound dressing with remarkable antibacterial and wound healing activities. International Journal of Biological Macromolecules, 2020, 149: 467-476.
27) KHABAROV, Vladimir N.; BOYKOV, Petr Ya; SELYANTN, Mikhail A. Hyaluronic acid: Production, properties, application in biology and medicine. John Wiley & Sons, 2014.
28) KOMUR, B., et al. Starch/PCI, composite nanofibers by co-axial electrospinning technique for biomedical applications. Biomedical engineering online, 2017, 16.1: 1-13.
29) REN, Ke, et al. Electrospun PCL/gelatin composite nanofiber structures for effective guided bone regeneration membranes. Materials Science and Engineering: C, 2017, 78: 324-332.
30) CHARERNSRIWILAIWAT, Natthan, et al. Electrospun chitosan/polyvinyl alcohol nanofibre mats for wound healing. International Wound Journal, 2014, 11.2: 215-222.

1. Means for wound healing based on hyaluronic acid derivatives, comprising nanofibers containing:
a crosslinked photocurable ester derivative of hyaluronic acid or a pharmaceutically acceptable salt thereof, wherein at least two ester groups of photocurable ester derivative of hyaluronic acid or a pharmaceutically acceptable salt thereof of general formula I 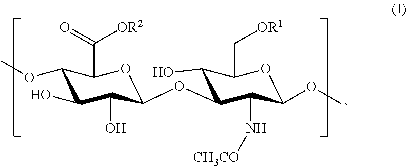
where
R¹ is independently H or COCHCH furyl,
R² is H⁺ or pharmaceutically acceptable salt,
and weight average molecular weight thereof is in the range from 82,000 g/mol to
110,000 g/mol and degree of substitution thereof is in the range from 4 to 20%, forms cyclobutane circle of general formula II, 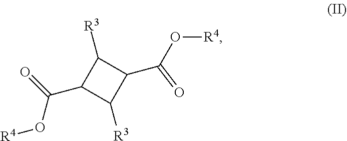
where
R³ is furyl and
R⁴ is main chain of hyaluronic acid or a pharmaceutically acceptable salt thereof,
hydrophobized derivative of hyaluronic acid or a pharmaceutically acceptable salt thereof of general formula III, 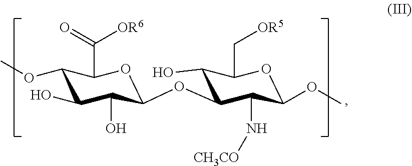
where
R⁵ is H or —C(═)C₁₂H₂₃,
R⁶ is H⁺ or pharmaceutically acceptable salt,
weight average molecular weight thereof is in the range from 300,000 g/mol to 350,000 g/mol and degree of substitution thereof is from 65% to 95%, and
polyethylene oxide of weight average molecular weight in the range from 300,000 g/mol to 900,000 g/mol.

2. The means of claim 1, wherein:
the degree of substitution of photocurable ester derivative of hyaluronic acid or a pharmaceutically acceptable salt thereof is in the range of from 5 to 10%,
the degree of substitution of hydrophobized hyaluronic acid derivative or a pharmaceutically acceptable salt thereof is in the range of from 65% to 80%, and
the weight average molecular weight of polyethylene oxide is from 400,000 g/mol to 600,000 g/mol.

3. The means of claim 1, wherein the nanofibers further contain at least one active agent, that comprises diagnostic agent and/or biologically active agent selected from a group containing antibiotics, antiallergics, antifungals, antineoplastics, antiphlogistics, antivirals, antioxidants, diagnostic agents or antiseptics or native hyaluronic acid or a pharmaceutically acceptable salt thereof, preferably biologically active agent is selected from a group containing: diclofenac, triclosan, octenidine, latanoprost, salicylic acid, gallic acid, ferulic acid, Ibuprofen, Naproxen, Cetirizine, quercetin, epicatechin, chrysin, luteolin, kurkumin, ciprofloxacin.

4. The means of claim 1, wherein the content of the crosslinked photocurable ester derivative of hyaluronic acid or the pharmaceutically acceptable salt thereof is from 15 wt. % to 75 wt. %, more preferably from 45 wt. % to 75 wt. %, the most preferably 48 wt. % relative to the total weight of nanofibers.

5. The means of claim 1, wherein a content of the hydrophobized derivative of hyaluronic acid or the pharmaceutically acceptable salt thereof is from 15 wt. % to 75 wt. %, more preferably from 45 wt. % to 75 wt. %, the most preferably 48 wt. %, relative to total weight of nanofibers.

6. The means of claim 1, wherein a content of polyethylene oxide is in the range from 3.5 wt. % to 10 wt. %, more preferably from 4 wt. % to 5 wt. %, the most preferably 4 wt. % relative to total weight of nanofibers.

7. The means of claim 1, wherein a content of the biologically active agent is in the range from 0.01 to 10 wt. %, preferably from 0.1 to 5 wt. %, relative to total weight of nanofibers.

8. The means of claim 1, wherein the nanofibers have diameter in the range from 100 nm to 1 000 nm, preferably from 250 nm to 500 nm.

9. The means of claim 1, in a form of dry layer, the areal weight thereof is in the range from 1 to 100 g/m², preferably in the range from 1 to 20 g/m², more preferably in the range from 10 to 15 g/m².

10. The means of claim 1, wherein an absorption capacity thereof is in the range from 1000 to 3500%, more preferably from 1500 to 2500%, for at least 1 hour after soaking in aqueous solution.

11. The means of claim 1, wherein a porosity thereof is maintained for 72 hours after soaking in aqueous solution.

12. A method of manufacture of the means of claim 1, comprising:
a spinning solution containing mixture of water and water miscible polar solvent, a photocurable ester derivative of hyaluronic acid or a pharmaceutically acceptable salt thereof of general formula I, a hydrophobized derivative of hyaluronic acid or a pharmaceutically acceptable salt thereof of general formula III and polyethylene oxide is electrostatically spun to form nanofibers, the formed nanofibers being photocured by crosslinking using irradiation in UV range of wavelengths.

13. The method of claim 12, wherein the water content in the spinning solution is in the range from 30 to 50 vol. %, more preferably 50 vol. % and the water miscible polar solvent is in the range from 50 to 70 vol. %, more preferably 50 vol. % relative to total volume of the spinning solution.

14. The method of claim 13, wherein the spinning solution contains distilled water and isopropyl alcohol.

15. The method of claim 12, wherein the spinning solution further contains at least one biologically active agent.

16. The method of claim 12, wherein the spinning solution has weight concentration of dry matter from 2 to 5 wt. %, preferably 3 wt. %, wherein the weight content in dry matter
of the photocurable ester derivative of hyaluronic acid or the pharmaceutically acceptable salt thereof being from 15 wt. % to 75 wt. %, more preferably from 45 wt. % to 75 wt. %, the most preferably 48 wt. %,
of the hydrophobized derivative of hyaluronic acid or the pharmaceutically acceptable salt thereof being from 15 wt. % to 75 wt. %, more preferably from 45 wt. % to 75 wt. %, the most preferably 48 wt. %,
polyethylene oxide being in the range from 4 wt. % to 10 wt. %, more preferably from 4 wt. % to 5 wt. %, the most preferably 4 wt. %.

17. The method of claim 15, wherein the weight proportion of biologically active compound in dry matter is in the range 0.01 to 10 wt. %, preferably 0.1 to 5 wt. %.

18. The method of claim 12, wherein the crosslinking by UV irradiation takes place for a period of 50 to 90 minutes, preferably 60 minutes.

19. A product comprising the means of claim 1, the product being for use in cosmetics, medicine or regenerative medicine, preferably in wound care or as a part of a patch for external or internal use.
